SEQUENCE LISTING (15 sequences)

SEQ ID NO: 1 - protein, 302 aa, Saccharomyces cerevisiae
MVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRSAQLGASMMGMAWASVFLCIVEFILLVFWSVRARLASTYSIDNSRYRTSSRWNPFHREKEQATDPILTATGPEDMQQSASIVGPSSNANPVTATAATENQPKGINFFTIRKSHERPDDVSV

SEQ ID NO: 2 - protein, 809 aa, Homo sapiens
MASPGHPAKSPRQPPAPRRDPFDAARGADFDHVYSGVVNLSTENIYSFNYTSQPDQVTAVRVYVNSSSENLNYPVLVVVRQQKEVLSWQVPLLFQGLYQRSYNYQEVSRTLCPSEATNETGPLQQLIFVDVASMAPLGAQYKLLVTKLKHFQLRTNVAFHFTASPSQPQYFLYKFPKDVDSVIIKVVSEMAYPCSVVSVQNIMCPVYDLDHNVEFNGVYQSMTKKAAITLQKKDFPGEQFFVVFVIKPEDYACGGSFFIQEKENQTWNLQRKKNLEVTIVPSIKESVYVKSSLFSVFIFLSFYLGCLLVGFVHYLRFQRKSIDGSFGSNDGSGNMVASHPIAASTPEGSNYGTIDESSSSPGRQMSSSDGGPPGQSDTDSSVEESDFDTMPDIESDKNIIRTKMFLYLSDLSRKDRRIVSKKYKIYFWNIITIAVFYALPVIQLVITYQTVVNVTGNQDICYYNFLCAHPLGVLSAFNNILSNLGHVLLGFLFLLIVLRRDILHRRALEAKDIFAVEYGIPKHFGLFYAMGIALMMEGVLSACYHVCPNYSNFQFDTSFMYMIAGLCMLKLYQTRHPDINASAYSAYASFAVVIMVTVLGVVFGKNDVWFWVIFSAIHVLASLALSTQIYYMGRFKIDLGIFRRAAMVFYTDCIQQCSRPLYMDRMVLLVVGNLVNWSFALFGLIYRPRDFASYMLGIFICNLLLYLAFYIIMKLRSSEKVLPVPLFCIVATAVMWAAALYFFFQNLSSWEGTPAESREKNRECILLDFFDDHDIWHFLSATALFFSFLVLLTLDDDLDVVRRDQIPVF

SEQ ID NO: 3 - protein, 16 aa, synthetic construct
SGGGGSGGGGSGGGGS

SEQ ID NO: 4 - protein, 182 aa, Saccharomyces cerevisiae
MVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRS

SEQ ID NO: 5 - protein, 832 aa, Mus musculus
MIAWRLPLCVLLVASVESHLGALGPKNVSQKDAEFERTYADDVNSELVNIYTFNHTVTRNRTEGVRVSVNVLNKQKGAPLLFVVRQKEAVVSFQVPLILRGLYQRKYLYQKVERTLCQPPTKNESEIQFFYVDVSTLSPVNTTYQLRVNRVDNFVLRTGELFTFNTTAAQPQYFKYEFPDGVDSVIVKVTSKKAFPCSVISIQDVLCPVYDLDNNVAFIGMYQTMTKKAAITVQRKDFPSNSFYVVVVVKTEDQACGGSLPFYPFVEDEPVDQGHRQKTLSVLVSQAVTSEAYVGGMLFCLGIFLSFYLLTVLLACWENWRQRKKTLLLAIDRACPESGHARVLADSFPGSAPYEGYNYGSFENGSGSTDGLVESAGSGDLSYSYQDRSFDAVGPRPRLDSMSSVEEDDYDTLTDIDSDKNVIRTKQYLCVADLARKDKRVLRKKYQIYFWNIATIAVFYALPVVQLVITYQTVVNVTGNQDICYYNFLCAHPLGNLSAFNNILSNLGYILLGLLFLLIILQREINHNRALLRNDLYALECGIPKHFGLFYAMGTALMMEGLLSACYHVCPNYTNFQFDTSFMYMIAGLCMLKLYQKRHPDINASAYSAYACLAIVIFFSVLGVVFGKGNTAFWIVFSVIHIISTLLLSTQLYYMGRWKLDSGIFRRILHVLYTDCIRQCSGPLYTDRMVLLVMGNIINWSLAAYGLIMRPNDFASYLLAIGICNLLLYFAFYIIMKLRSGERIKLIPLLCIVCTSVVWGFALFFFFQGLSTWQKTPAESREHNRDCILLDFFDDHDIWHFLSSIAMFGSFLVLLTLDDDLDTVQRDKIYVF

SEQ ID NO: 6 - DNA, 808 bp, synthetic construct
  source 1..808: mol_type = other DNA
taatacgactcactatagggagacccaagcttggtaccgagctcggatccgccaccatgaagaccttaattcttgccgttgcattagtctactgcgccactgttcattgccagatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatagtggacaagcatcaagcacgcagcaacaagcacgagcaatgagccagcctagccacacccccacgggaaacagcagtgattaacctttagcaataaacgaaagtttaaccaagctatactaaccccagggttggtcaatttcgtgccagccacaccgtcacacgattaacccaagtc

SEQ ID NO: 7 - protein, 1127 aa, synthetic construct
MASPGHPAKSPRQPPAPRRDPFDAARGADFDHVYSGVVNLSTENIYSFNYTSQPDQVTAVRVYVNSSSENLNYPVLVVVRQQKEVLSWQVPLLFQGLYQRSYNYQEVSRTLCPSEATNETGPLQQLIFVDVASMAPLGAQYKLLVTKLKHFQLRTNVAFHFTASPSQPQYFLYKFPKDVDSVIIKVVSEMAYPCSVVSVQNIMCPVYDLDHNVEFNGVYQSMTKKAAITLQKKDFPGEQFFVVFVIKPEDYACGGSFFIQEKENQTWNLQRKKNLEVTIVPSIKESVYVKSSLFSVFIFLSFYLGCLLVGFVHYLRFQRKSIDGSFGSNDGSGNMVASHPIAASTPEGSNYGTIDESSSSPGRQMSSSDGGPPGQSDTDSSVEESDFDTMPDIESDKNIIRTKMFLYLSDLSRKDRRIVSKKYKIYFWNIITIAVFYALPVIQLVITYQTVVNVTGNQDICYYNFLCAHPLGVLSAFNNILSNLGHVLLGFLFLLIVLRRDILHRRALEAKDIFAVEYGIPKHFGLFYAMGIALMMEGVLSACYHVCPNYSNFQFDTSFMYMIAGLCMLKLYQTRHPDINASAYSAYASFAVVIMVTVLGVVFGKNDVWFWVIFSAIHVLASLALSTQIYYMGRFKIDLGIFRRAAMVFYTDCIQQCSRPLYMDRMVLLVVGNLVNWSFALFGLIYRPRDFASYMLGIFICNLLLYLAFYIIMKLRSSEKVLPVPLFCIVATAVMWAAALYFFFQNLSSWEGTPAESREKNRECILLDFFDDHDIWHFLSATALFFSFLVLLTLDDDLDVVRRDQIPVFGGGGSGGGGSGGGGSMVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRSAQLGASMMGMAWASVFLCIVEFILLVFWSVRARLASTYSIDNSRYRTSSRWNPFHREKEQATDPILTATGPEDMQQSASIVGPSSNANPVTATAATENQPKGINFFTIRKSHERPDDVSVS

SEQ ID NO: 8 - protein, 1007 aa, synthetic construct
MVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRSSGGGGSGGGGSGGGGSMASPGHPAKSPRQPPAPRRDPFDAARGADFDHVYSGVVNLSTENIYSFNYTSQPDQVTAVRVYVNSSSENLNYPVLVVVRQQKEVLSWQVPLLFQGLYQRSYNYQEVSRTLCPSEATNETGPLQQLIFVDVASMAPLGAQYKLLVTKLKHFQLRTNVAFHFTASPSQPQYFLYKFPKDVDSVIIKVVSEMAYPCSVVSVQNIMCPVYDLDHNVEFNGVYQSMTKKAAITLQKKDFPGEQFFVVFVIKPEDYACGGSFFIQEKENQTWNLQRKKNLEVTIVPSIKESVYVKSSLFSVFIFLSFYLGCLLVGFVHYLRFQRKSIDGSFGSNDGSGNMVASHPIAASTPEGSNYGTIDESSSSPGRQMSSSDGGPPGQSDTDSSVEESDFDTMPDIESDKNIIRTKMFLYLSDLSRKDRRIVSKKYKIYFWNIITIAVFYALPVIQLVITYQTVVNVTGNQDICYYNFLCAHPLGVLSAFNNILSNLGHVLLGFLFLLIVLRRDILHRRALEAKDIFAVEYGIPKHFGLFYAMGIALMMEGVLSACYHVCPNYSNFQFDTSFMYMIAGLCMLKLYQTRHPDINASAYSAYASFAVVIMVTVLGVVFGKNDVWFWVIFSAIHVLASLALSTQIYYMGRFKIDLGIFRRAAMVFYTDCIQQCSRPLYMDRMVLLVVGNLVNWSFALFGLIYRPRDFASYMLGIFICNLLLYLAFYIIMKLRSSEKVLPVPLFCIVATAVMWAAALYFFFQNLSSWEGTPAESREKNRECILLDFFDDHDIWHFLSATALFFSFLVLLTLDDDLDVVRRDQIPVF

SEQ ID NO: 9 - protein, 1150 aa, synthetic construct
MVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRSAQLGASMMGMAWASVFLCIVEFILLVFWSVRARLASTYSIDNSRYRTSSRWNPFHREKEQATDPILTATGPEDMQQSASIVGPSSNANPVTATAATENQPKGINFFTIRKSHERPDDVSVSGGGGSGGGGSGGGGSMIAWRLPLCVLLVASVESHLGALGPKNVSQKDAEFERTYADDVNSELVNIYTFNHTVTRNRTEGVRVSVNVLNKQKGAPLLFVVRQKEAVVSFQVPLILRGLYQRKYLYQKVERTLCQPPTKNESEIQFFYVDVSTLSPVNTTYQLRVNRVDNFVLRTGELFTFNTTAAQPQYFKYEFPDGVDSVIVKVTSKKAFPCSVISIQDVLCPVYDLDNNVAFIGMYQTMTKKAAITVQRKDFPSNSFYVVVVVKTEDQACGGSLPFYPFVEDEPVDQGHRQKTLSVLVSQAVTSEAYVGGMLFCLGIFLSFYLLTVLLACWENWRQRKKTLLLAIDRACPESGHARVLADSFPGSAPYEGYNYGSFENGSGSTDGLVESAGSGDLSYSYQDRSFDAVGPRPRLDSMSSVEEDDYDTLTDIDSDKNVIRTKQYLCVADLARKDKRVLRKKYQIYFWNIATIAVFYALPVVQLVITYQTVVNVTGNQDICYYNFLCAHPLGNLSAFNNILSNLGYILLGLLFLLIILQREINHNRALLRNDLYALECGIPKHFGLFYAMGTALMMEGLLSACYHVCPNYTNFQFDTSFMYMIAGLCMLKLYQKRHPDINASAYSAYACLAIVIFFSVLGVVFGKGNTAFWIVFSVIHIISTLLLSTQLYYMGRWKLDSGIFRRILHVLYTDCIRQCSGPLYTDRMVLLVMGNIINWSLAAYGLIMRPNDFASYLLAIGICNLLLYFAFYIIMKLRSGERIKLIPLLCIVCTSVVWGFALFFFFQGLSTWQKTPAESREHNRDCILLDFFDDHDIWHFLSSIAMFGSFLVLLTLDDDLDTVQRDKIYVF

SEQ ID NO: 10 - protein, 1030 aa, synthetic construct
MVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRSSGGGGSGGGGSGGGGSMIAWRLPLCVLLVASVESHLGALGPKNVSQKDAEFERTYADDVNSELVNIYTFNHTVTRNRTEGVRVSVNVLNKQKGAPLLFVVRQKEAVVSFQVPLILRGLYQRKYLYQKVERTLCQPPTKNESEIQFFYVDVSTLSPVNTTYQLRVNRVDNFVLRTGELFTFNTTAAQPQYFKYEFPDGVDSVIVKVTSKKAFPCSVISIQDVLCPVYDLDNNVAFIGMYQTMTKKAAITVQRKDFPSNSFYVVVVVKTEDQACGGSLPFYPFVEDEPVDQGHRQKTLSVLVSQAVTSEAYVGGMLFCLGIFLSFYLLTVLLACWENWRQRKKTLLLAIDRACPESGHARVLADSFPGSAPYEGYNYGSFENGSGSTDGLVESAGSGDLSYSYQDRSFDAVGPRPRLDSMSSVEEDDYDTLTDIDSDKNVIRTKQYLCVADLARKDKRVLRKKYQIYFWNIATIAVFYALPVVQLVITYQTVVNVTGNQDICYYNFLCAHPLGNLSAFNNILSNLGYILLGLLFLLIILQREINHNRALLRNDLYALECGIPKHFGLFYAMGTALMMEGLLSACYHVCPNYTNFQFDTSFMYMIAGLCMLKLYQKRHPDINASAYSAYACLAIVIFFSVLGVVFGKGNTAFWIVFSVIHIISTLLLSTQLYYMGRWKLDSGIFRRILHVLYTDCIRQCSGPLYTDRMVLLVMGNIINWSLAAYGLIMRPNDFASYLLAIGICNLLLYFAFYIIMKLRSGERIKLIPLLCIVCTSVVWGFALFFFFQGLSTWQKTPAESREHNRDCILLDFFDDHDIWHFLSSIAMFGSFLVLLTLDDDLDTVQRDKIYVF

SEQ ID NO: 11 - DNA, 20 bp, synthetic construct
  source 1..20: mol_type = other DNA
ttgccggtggtggagatgaa

SEQ ID NO: 12 - DNA, 20 bp, Bacteriophage t7
  source 1..20: mol_type = genomic DNA
taatacgactcactataggg

SEQ ID NO: 13 - DNA, 25 bp, synthetic construct
  source 1..25: mol_type = other DNA
tactggcttatcgaaattaatacga

SEQ ID NO: 14 - DNA, 45 bp, synthetic construct
  source 1..45: mol_type = other DNA
tctagataatacgactcactataggattctgaaacaaactggaca

SEQ ID NO: 15 - DNA, 45 bp, synthetic construct
  source 1..45: mol_type = other DNA
tctagataatacgactcactataggttccacagggtccctgttac

(19) United States
(12) Patent Application Publication
(10) Pub. No.: US 2026/0199512 A1
(43) Pub. Date: Jul. 16, 2026

(54) METHODS FOR ACTIVELY LOADING THERAPEUTIC NUCLEIC ACID MOLECULES INTO MODIFIED EXTRACELLULAR VESICLES FOR THE TARGETED DELIVERY TO CELLS

(71) Applicant: Mercury Bio, Inc., Santa Fe, NM (US)

(72) Inventors: Richard T. Sayre, Los Alamos, NM (US); Tatiana Vinogradova-Shah, Rochester, NY (US); Elena Sineva, Bernalillo, NM (US)

(21) Appl. No.: 19/138,527

(22) PCT Filed: Dec. 12, 2023
(86) PCT No.: PCT/US2023/083644

§ 371 Date: Jun. 12, 2025

Related U.S. Application Data
(60) Provisional application No. 63/431,853, filed on Dec. 12, 2022.

(51) Int. Cl. A61K 47/69; C07K 14/705; C12N 1/18; C12R 1/85

(52) U.S. Cl. CPC A61K 47/6901; C07K 14/705; C12N 1/18; C07K 2319/80; C12R 2001/85

(57) ABSTRACT

Systems, methods and compositions for the production and use of Saccharomyces-generated extracellular vesicles having a membrane-bound nucleic acid transport system adapted to actively transport one or more nucleic acids into the lumen of the vesicle.

### CROSS-REFERENCE TO RELATED APPLICATIONS

This International PCT application claims the benefit of and priority to U.S. Provisional Application No. 63/431,853, filed Dec. 12, 2022, the specification, claims and drawings of which are incorporated herein by reference in their entirety.

### SEQUENCE LISTING

The instant application contains contents of the electronic sequence listing (90355.00101-Sequence-Listing.xml; Size: 21,229 bytes; and Date of Creation: Dec. 12, 2023) is herein incorporated by reference in its entirety.

### TECHNICAL FIELD

The present invention is directed to novel methods, systems, and compositions to facilitate the active loading of therapeutic nucleic acid compositions into yeast-generated extracellular vesicles (yEVs) and their methods of use in treating various diseases or conditions.

### BACKGROUND

One of the major challenges facing the use of therapeutic molecules and compounds, also sometimes referred to as drugs, therapeutics, or therapeutic compositions, to control diseases and genetic disorders is an effective means to deliver the drug to a targeted tissue while avoiding its degradation or elimination from the body. Recently, lipid encased nanovesicles have proven to be an effective means to deliver therapeutic molecules to human cells while protecting their cargo from degradation. The use of lipid nanovesicles to deliver mRNA to cells has proven to be an efficient means to vaccinate humans against SARS-CoV-2. However, the artificial lipids frequently used to make these nanovesicles are not tissue or cell specific and are not well tolerated by humans limiting their applications to non-therapeutic purposes.

An alternative, natural lipid nanovesicle that has also been shown to package RNA, proteins and other small molecules are extracellular vesicles (EVs) or exosomes produced and released from the surface of eukaryotic cells. Most eukaryotic EVs are well tolerated but have limitations due to the heterogeneity of cell types from which they originate resulting in broad distributions of particle size, composition and heterogeneous cargo. Furthermore, EVs or exosomes produced in human cell lines can potentially be contaminated by human pathogens such as viruses, contain off-target RNAs and are expensive to produce in culture.

An alternative platform for producing more uniform EVs that package therapeutic molecules is the production of EVs in single celled eukaryotic organisms (non-human) having uniform genetic traits. Examples of single-celled organisms that can be engineered to deliver therapeutic molecules include the yeast, such as Saccharomyces cerevisiae and S. boulardii. Significantly, these yeast strains are well tolerated by humans being used in the manufacture of foods and beverages consumed by humans. Significantly, S. boulardii has been safely used as human probiotic for over 65 years. More specifically, S. boulardii has several traits that are ideal to produce EVs for delivery of therapeutics to humans. S. boulardii produces EVs that package RNA, proteins, and other small molecules. In addition, the genome of S. boulardii is available and S. boulardii can be genetically engineered and/or have its genome edited for the purpose of modifying EV targeting or cargo. S. boulardii can also be grown at large scale in fermenters and has a shelf life of over a year at room temperature when freeze-dried. Yeast EVs are also safe. They lack RNA species that can alter protein expression in human cells and do not induce the production of antibodies. Finally, yeast EVs can be delivered to multiple organs in the body either by injection or oral delivery. These traits open the possibility of using S. boulardii as a complete, single celled system for the large-scale production and packaging of therapeutic molecules into EVs for delivery to humans.

Several traditional methods have been developed to load therapeutic composition into EVs for delivery to target cells or tissues. These methods for encapsulating cargo into EVs can be roughly divided into two types: cell-based loading methods and non-cell-based loading methods. In the cell-based loading approach, cargo molecules are usually produced in the donor cells first. After being packaged into EVs, the cargo molecules can be collected in an EV-carrying manner for therapeutic use. Non-cell-based loading approach involves directly loading chemical or biomolecules into isolated EVs through electroporation, sonication, incubation, and/or transfection.

However, each of these methods involve specific technical and commercial limitations. For example, passive incubation loading typically has low loading efficiencies, while transfection relies on transfection efficiency, which is always variable, and may further alter the structure of the EVs. Methods such as electroporation, sonication, and freeze thaw disrupt the EVs structure including the topological orientation of cell-specific targeting molecules, increase EV instability and can further result in undesired EV aggregation. Finally, current cell and non-cell-based methods of EV loading lack the ability to precisely, selectively, and uniformly load specific molecules into EVs so that they can be more effectively dosed for therapeutic applications. As such, there exist a long-felt need for an efficient, and commercially viable method to load therapeutic compositions, and in particular therapeutic molecules and nucleic acids precisely and uniformly into EVs structures, all while causing minimal disruption to their structure and aggregation patterns.

### SUMMARY OF THE INVENTION

The present invention is directed to novel methods, systems, and compositions for actively loading nucleic acids, and in particular therapeutic nucleic acids into a yeast-derived extracellular vesicle (yEV), wherein the yEV can deliver the nucleic acids to a target cell thereby treating a disease or condition or generating some physiological or other effect.

In another aspect, the present invention includes systems and compositions to genetically modify yeast cells, and preferably a Saccharomyces yeast cell, to express one or more heterologous fusion peptides configured to facilitate the active transport of nucleic acids into a yEV. In one preferred aspect, the current invention includes novel constructs for the heterologous expression of one or more fusion peptides in yeast that are adapted to facilitate active transport of therapeutic nucleic acid compositions into a yEV.

Another aspect of the invention includes methods and compositions for the co-expression of EV surface displayed receptor-specific ligands anchored to EV membrane proteins. In this preferred aspect, genetically modified yeast cells are adapted to heterologously express and present on the surface of EVs receptor-specific ligands anchored to EV membrane proteins as a fusion peptide.

Additional aspects of the invention will be evident from the specification, figures, and claims provided herein.

### BRIEF DESCRIPTION OF THE FIGURES

FIG. 1A-B. Model of the directional transport of nucleic acids by SidT1 protein inserted in the membrane of yEVs. (A) Fusion of SidT1 to the N-terminus of Sur7 protein allows for the positioning of the N-terminus of SidT1 on the outer surface of the membrane and unrestrained folding of N terminal domain of SidT1 which supports the inward transport of nucleic acids. (B) Fusion of SidT2 to the C-terminal of Sur7 protein allows for the positioning of the main active cytosolic domain to be located on the outer surface of the membrane and supports the directional transport of nucleic acids.

FIG. 2A-B. ssDNA uptake by yEVs expressing a gene fusion of the yeast Sur7 protein with the SidT1 or SidT2 proteins. A. Relative loading of eGFP-Alexa488 ssDNA oligo into yEVs. B. ssDNA concentrations in SidT1-Sur7 and Sur7f-SidT2 yEVs. The labels on the graphs correspond to the names of construct expressed by yEVs.

FIG. 3 nLuc mRNA BODIPY488 transport into yEVs expressing SidT1-Sur7 or into wild-type yEVs. The labels on the graphs correspond to the names of construct expressed by yEVs.

FIG. 4A-B. mRNA nLuc transport into yEVs expressing the Sur7f-SidT2 fusion protein: A. Relative yEV load of nLuc mRNA determined by qPCR B. Relative fluorescent signal of nLuc mRNA in yEVs. Absolute nLuc mRNA concentration was ~1 nM.

FIG. 5 Transport of 500 bp dsRNA into yEVs expressing the SidT1-Sur7 or Sur7f-SidT2 proteins or wild-type yEVs. The labels on the graphs correspond to the names of construct expressed by yEVs.

FIG. 6. siRNA-Cy3 transport into yEVs expressing the Sur7f-SidT2 protein. The labels on the graphs correspond to the names of construct expressed by yEVs.

### DETAILED DESCRIPTION OF THE INVENTION

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference. The terminology used herein is for describing particular embodiments and is not intended to be limiting. As used herein, the singular forms “a,” “and” and “the” include plural referents unless the content and context clearly dictate otherwise. Thus, for example, a reference to “a” or “the” marker may include a combination of two or more such markers. Unless defined otherwise, all scientific and technical terms are to be understood as having the same meaning as commonly used in the art to which they pertain. For the purposes of the present invention, the following terms are defined above.

The present invention is directed to EVs derived from Saccharomyces, such as Saccharomyces cerevisiae or Saccharomyces boulardii engineered to actively load nucleic acids into the yEV's luminal space, which can be isolated and administered to a subject in need thereof to treat a disease or condition. In one aspect, the invention describes novel systems, methods and compositions for actively loading nucleic acids, such as therapeutic RNA and/or DNA molecules into a yEVs.

In another aspect, the invention describes novel systems, methods and compositions for actively loading RNA or DNA compositions, such as siRNAs, ssRNA, dsRNAs, microRNAs, mRNAs, lncRNAs, circular RNAs, self-replicating or amplifying RNAs, chemically modified RNAs, and linear or circular DNA into yeast yEVs using a nucleic acid transporter protein(s), such as a transporter from the SID1 family, anchored to an EV membrane protein.

In another preferred aspect, the invention includes a genetically modified yeast cell configured to express a heterologous fusion peptide for actively loading therapeutic or other nucleic acids into an EV. In this preferred embodiment, this may include an EV membrane protein anchor fused to a nucleic acid transporter protein. In another preferred embodiment, a heterologous fusion peptide for actively loading therapeutic nucleic acids into an EV may include: 1) an EV-localized S. cerevisiae protein such as the SUR7 membrane protein; 2) the ATP-dependent SIDT1 or SIDT2 and related family of lysosomal RNA or DNA membrane transporting proteins as found in humans.

In one preferred embodiment, the invention describes novel systems, methods and compositions actively loading therapeutic RNA and/or DNA molecules into yeast yEVs nucleic acid transport system anchored to the membrane of said yEV and adapted to actively transport one or more nucleic acid compositions into said yEV. The nucleic acid transport system of the invention includes a chimeric, or fusion peptide having a first and second domain. In this preferred embodiment, the fusion peptide of the invention includes a first domain comprising a comprising nucleic acid transporter, and a second domain comprising a yEV membrane protein anchor.

The first and second domain preferably being joined by a linker, such as a linker peptide (SEQ ID NO. 3) In this embodiment of the invention, wherein said linker peptide comprises a peptide adapted to allow proper conformational folding of the N- and C-terminal portions of the fusion peptide. Notably, as described below the position of the first and second domains of the fusion peptide are variable with respect to their position. For example, in one embodiment, the first domain is positioned at the N-terminal portion of the fusion peptide, and the second domain is positioned at the C-terminal portion of the fusion peptide. In an alternative embodiment, the first domain is positioned at the C-terminal portion of the fusion peptide, and the second domain is positioned at the N-terminal portion of the fusion peptide.

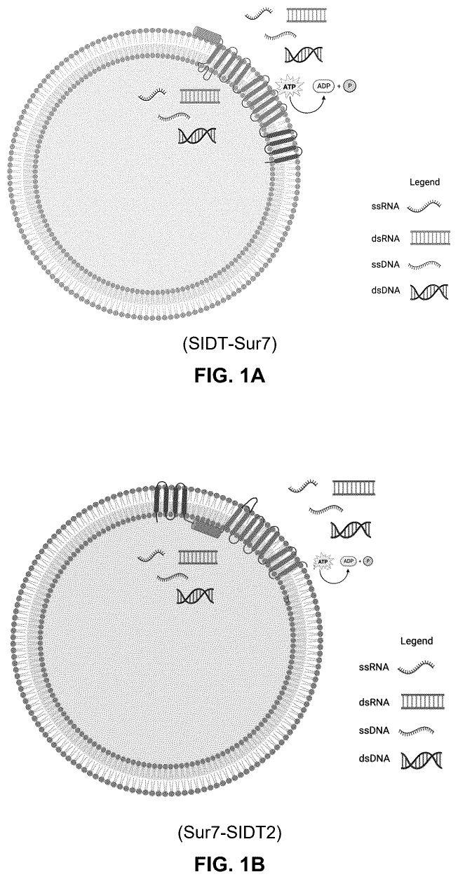

As generally shown in FIG. 1A, the configuration of the first and second domains of the invention allows the membrane protein anchor of the fusion peptide to be bound to the membrane of the yEV in such an orientation relative to the membrane of the yEV, such that the N-terminal portion of the nucleic acid transporter is positioned on the outside the EV membrane allowing the directional transport of nucleic acids into the lumen of the yEV. Also as shown in FIG. 1 the configuration of first and second domains of the invention supports the native conformation of nucleic acid transporter necessary for vectoral transport of nucleic acids into the yEV.

Again, as generally showing in FIG. 1, the nucleic acid transport system of the invention can include a fusion peptide having a first and second domain, wherein the first domain includes EV membrane protein anchor comprises an EV localized Saccharomyces cerevisiae SUR7 peptide, or other suitable EV-specific transmembrane protein, or a fragment or variant thereof. In another embodiment, the nucleic acid transport system of the invention can include a fusion peptide having a first and second domain, wherein the first domain includes an EV membrane protein anchor selected from: SEQ ID NO's. 1, 4, or a fragment or variant thereof. In another embodiment, the nucleic acid transport system of the invention can include a fusion peptide having a first and second domain, wherein the first domain includes an EV membrane protein anchor comprises a peptide having at least 80% sequence identity to SEQ ID NO. 1. As noted above, this first domain can be position at the N- or C-terminal portion of the fusion peptide and can be anchored in the outer membrane of a yEV, which as described above allows for native orientation and configuration of the N-terminal portion of the second domain.

Again, as generally showing in FIG. 1, the nucleic acid transport system of the invention can include a fusion peptide having a first and second domain, wherein the second domain includes a nucleic acid transporter selected from: SIDT1, SIDT2, or a fragment or variant thereof. In one preferred embodiment, the signal peptide signal portion of SIDT1 can be removed or disrupted. In a specific embodiment, the first 19 residues of SIDT1 as described in SEQ ID NO. 1, can be removed to decrease the chances of off-target localization to other than a yEV. In another embodiment, the nucleic acid transport system of the invention can include a fusion peptide having a first and second domain, wherein the second domain includes nucleic acid transporter is selected from: SEQ ID NO's. 2, 5, or a fragment or variant thereof. In another embodiment, the nucleic acid transport system of the invention can include a fusion peptide having a first and second domain, wherein the second domain includes nucleic acid transporter is includes a peptide having at least 80% sequence identity to SEQ ID NO. 2, or a peptide having at least 80% sequence identity to SEQ ID NO. 5.

In further embodiments, the nucleic acid transport system of the invention a fusion peptide bound to the membrane of a yEV, and preferably a yEV from Saccharomyces cerevisiae, or Saccharomyces boulardii, the fusion peptide having a first domain at the N-terminal position comprising an EV membrane protein anchor according to SEQ ID NO. 1, or a fragment or variant thereof, and a second domain at the C-terminal position comprising nucleic acid transporter according to SEQ ID NO. 5, or a fragment or variant thereof. The first and second domain being joined in this embodiment by a peptide linker, and preferably a peptide linker according to SEQ ID NO. 3

In still further embodiments, the nucleic acid transport system of the invention includes a fusion peptide bound to the membrane of a yEV, and preferably a yEV from Saccharomyces cerevisiae, or Saccharomyces boulardii, the fusion peptide having a first domain at the C-terminal position comprising an EV membrane protein anchor according to SEQ ID NO. 1, or a fragment or variant thereof, and a second domain at the N-terminal position comprising nucleic acid transporter according to SEQ ID NO. 2. The first and second domain being joined in this embodiment by a peptide linker, and preferably a peptide linker according to SEQ ID NO. 3

In further embodiments, the nucleic acid transport system of the invention a includes a heterologous nucleotide sequence, operably linked to a promoter, encoding fusion peptide having a first domain encoding a yEV membrane protein anchor, and a second domain nucleic acid transporter as described herein. Noting that the definition of first and second do not dictate their placement in series or with respect to other elements. In one preferred embodiment, the heterologous nucleotide sequence encoding fusion peptide can include a nucleotide sequence, forming an expression cassette that can be operably linked to a promoter, such as a T7 promoter (SEQ ID NO. 12). In one embodiment the expression cassette and promoter can include the nucleotide sequence according to SEQ ID NO. 7, or SEQ ID NO. 9. Notably, all nucleotide sequences encoding the fusion peptide, or other heterologously expressed peptides of the invention can be codon optimized for expression in yeast, and preferably Saccharomyces boulardii. In another preferred embodiment, yeast transgene constructs can be cloned into yeast/E. coli shuttle vectors such as pRS416 and pRS426 for expression of the transgene constructs using the appropriate yeast gene promoter/terminator, such as TDH3p/CYC1t, all of the above being readily known and understood by those skilled in the art.

As noted above, a yeast cell, and preferably a Saccharomyces cerevisiae, or Saccharomyces boulardii can be transformed to express heterologous nucleic acid transport system comprising a fusion peptide having a first and second domain as described herein. In this embodiment, the fusion peptide can be heterologously expressed in the transformed yeast cell and incorporated into yEVs produced by the cell. These transformed yeast cells can be cultured, preferably under conditions that promote extracellular vesicle generation and isolating the EVs from culture, as well as the uptake of nucleic acids.

The yEVs of the invention can further be isolated from the yeast cells and combined with a pharmaceutically acceptable carrier forming a pharmaceutical composition. Such a pharmaceutical composition can be part of a kit that includes a container, a metered dose of the pharmaceutical composition, and optionally instructions for use. The pharmaceutical compositions of the invention can be administered to a subject in need thereof. In this embodiment, a pharmaceutical composition containing a yEV containing a therapeutically effective amount of nucleic acids directed to treat or prevent a disease or condition can be administered to a subject in need thereof, and preferably a human subject.

In another embodiment, the invention is directed to genetically modified yeast cells adapted to heterologously express and present on the surface of EVs receptor-specific ligands anchored to EV membrane proteins as a fusion peptide. In another embodiment, cancer cell specific or enriched receptor ligands can be anchored to the EV membrane protein C- or N-terminus so that the ligands are displayed on the surface of the EV. More specifically surface displayed ligands to target EVs to P-selectin receptors which are over-expressed in blood vessels of variety of cancers including ovarian cancer; NRP1 receptor, follicle-stimulating hormone (FSH) receptor FSHR; epidermal growth factor (EGF) receptor. The examples of receptor-specific ligands, also referred to generally as receptor-binding ligands used for fusion are C-end class peptides for binding NRP1 receptor; AEYLR-small peptide binding EGFR; and small peptide FSH33-53 for binding FSHR.

In another aspect, the EVs of the invention may be modified to display surface ligands that target delivery of the EVs to unique receptors displayed on the surfaces of targeted cell types. The receptor-specific ligands may be fused or anchored to EV membrane proteins and displayed on the EV exterior surface. Examples of EVs displaying surface ligands for directed transport via target cell receptors are described by Sayre et al., in PCT/US2022/014958, which is incorporated herein by reference.

In another embodiment, the EVs of the invention may be modified to display heterologous antibodies, and preferably nanobodies anchored to yEV membrane anchor proteins and displayed on the outer surface of the yEV. In a preferred embodiment, the anchored nanobodies can recognize and bind to a cognate antigen displayed on a target cell, and preferably a target human cell. In this manner, the yEVs of the invention can be directed to one or more target cells or tissues, and may further facilitate the delivery of therapeutic nucleic acids as generally described herein to target cells that are implicated in a disease or other condition.

The term “endogenous” gene or protein means that said gene or protein is expressed from a gene naturally found in the genome of a eukaryotic cell. The term “heterologous” gene or protein means that said gene or protein is not expressed from a gene naturally found in the genome of a eukaryotic cell. As used herein, the term “gene” or “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

As used herein, the phrase “expression,” “gene expression” or “protein expression,” such as the level of includes any information pertaining to the amount of gene transcript or protein present in a sample, in a cell, in a patient, secreted in a sample, and secreted from a cell as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information.” Such information may be given in the form of amounts per cell, amounts relative to a control gene or protein, in unitless measures, etc.

The term “fusion protein” as used herein is used as it is in the art. Namely, the fusion proteins used in the methods and compositions of the present invention involve two separate proteins or protein domains that are linked by a covalent bond. In one embodiment, the covalent bond linking the two domains is an amine bond. In more specific embodiments, the anchor protein and the foreign protein is a fusion protein comprising a single-chain polypeptide. In even more specific embodiments, the single-chain polypeptide comprising the anchor protein and the foreign protein further comprises a linker peptide sequence. Any linker sequence can be used to covalently link the anchor protein and the foreign protein.

As used herein, the term “peptide linker(s),” “linker(s),” or “linker moiety” refers to a peptide or polypeptide sequence, e.g., a synthetic peptide or polypeptide sequence, which connects two domains in a linear amino acid sequence of a polypeptide chain. In one embodiment, the polypeptides of invention are encoded by nucleic acid molecules that encode peptide linkers which either directly or indirectly connect the anchor protein and foreign protein which make up the construct. These linkers may be interposed between the anchor protein and foreign protein. If the linker connects two protein moieties contiguously in the linear polypeptide sequence, it is referred to as a “direct” linkage. In contrast, the linkers may link the first protein moiety, i.e., anchor protein or foreign protein, to a binding moiety which is, in turn, linked to the second protein moiety, i.e., anchor protein or foreign protein, thereby forming an indirect linkage. Linkers are typically located at the N or C terminus of the protein moieties.

In one embodiment, the linker linking the anchor protein and the foreign protein is a peptide comprised of glycine (Gly) n, wherein n is an integer that is the same or higher than 1, 2, 3, 4, 5, 6, 7, 8, 9, 10. In another embodiment, the linker linking the anchor protein and the foreign protein is a gly-ser linker. As used herein, the term “gly-ser peptide linker” (GS) refers to a peptide comprising or consisting of glycine (G or Gly) and serine (Sor Ser) residues. Exemplary gly-ser peptide linkers comprise the amino acid sequence (Gly4 Ser)n or (Gly3 Ser)n. Another exemplary gly-ser peptide linker comprises the amino acid sequence S(Gly4 Ser)n wherein n is an integer that is the same or higher than 1, 2, 3, 4, 5, 6, 7, 8, 9, 10. In another embodiment, the linker linking the anchor protein and the foreign protein is a peptide comprising SEQ ID NO. 3.

As used herein, a “fragment” of a polypeptide refers to a single amino acid or a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. A “variant” include, a peptide having the same function as an identified peptide, while having a different sequence or being derived from a different organism than an identified peptide.

As used herein, a “fragment” of poly- or oligonucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g., 5, 6, 7, 8, 9, 10, 11 or 12 or more amino acids long, including each integer up to the full length of the polypeptide). Those skilled in the art are well versed in methods for selecting antigenic fragments of proteins.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “introducing,” “administered” or “administering”, as used herein, refers to any method of providing a composition of EVs to a patient such that the composition has its intended effect on the patient. In one embodiment, EVs may be introduced to a patient in vivo, while in other alternative embodiments, EVs may be introduced to subject cells in vitro which may then be administered to a patient in vivo.

The term “patient,” or “subject” as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

An expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “expression cassette” refers to a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may be chimeric, meaning that at least one of its components is heterologous with respect to at least one of its other components. The peptides of the invention of the present invention may be chimeric.

The expression cassette may also be one which is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus.

As used herein, a promoter region or promoter element refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

To accomplish delivery of therapeutic compositions, such as nucleic acids to target cells, the methods and compositions of the present invention comprise extracellular vesicles (EVs), and preferably EV generated from Saccharomyces, such as S. boulardii or S. cerevisiae. EVs generated from Saccharomyces spp. sometimes are also interchangeably referred to as Saccharomyces-generated extracellular vesicles, SEVGs, sometimes also referred to as yEVs, or yeast extracellular vesicles. The term extracellular vesicles are membranous vesicles released from cells. The extracellular vesicles of the methods and compositions of the invention are composed of lipid bilayers that can envelope and carry cargo in its interior. The lipid bilayer of the EVs may also include proteins embedded therein. In some embodiments, the yEVs of the compositions and methods of the present invention can be exosomes or ectosomes. As is well-known, exosomes are generally formed upon the endocytosis of multivesicular endosomes (MVEs) to form intraluminal vesicles (ILVs) which are subsequently released into the extracellular environment as exosomes, whereas ectosomes are assembled and released from the plasma membrane. Often, the primary structural feature distinguishing ectosomes and ectosomes is diameter. In some embodiments, the diameter of the yEVs are between about 30 nm to about 180 nm, between about 50 nm to about 200 nm, between about 75 nm to about 250 nm, between about 100 nm to about 300 nm, between about 125 nm to about 350 nm, between about 150 nm to about 400 nm, between about 175 nm to about 450 nm, between about 200 nm to about 500 nm, between about 250 nm to about 550 nm, between about 300 nm to about 600 nm, between about 350 nm to about between about 650 nm, between about 400 nm to about 700 nm, between about 450 nm to about 750 nm, between about 500 to about 800 nm, between about 550 nm to about 850 nm, between about 600 nm to about 900 nm, between about 650 nm to about 950 nm, between about 700 nm to about 1000 nm, between about 750 nm to about 1050 nm, between about 800 nm to about 1100 nm, between about 850 nm to about 1150 nm or between about 900 nm to about 1200 nm. Thus, exosomes may comprise components on their membrane surface, including but not limited to proteins, glycoproteins, proteoglycans, carbohydrates and lipids, which may be used to direct cargo into to exosome.

As understood by the disclosure herein, Saccharomyces is a single-celled organism, but the term “extracellular vesicle,” as it relates to the yEVs, refers to vesicles that are secreted from Saccharomyces into the local environment, such as, but not limited to cell culture medium and organisms that may have ingested or consumed or been administered the Saccharomyces secreting the vesicles containing a therapeutic composition. In one embodiment, the EVs are secreted from Saccharomyces cerevisiae or Saccharomyces boulardii.

The polynucleotides of the present invention may be in the form of RNA or in the form of DNA, which DNA includes cDNA, genomic DNA, and synthetic DNA. The DNA may be double-stranded or single-stranded, and if single stranded may be the coding strand or non-coding (antisense) strand. The coding sequence which encodes the peptides may be identical to the coding sequence shown in the sequence listing, or that of any of the deposited clones, or may be a different coding sequence which, as a result of the redundancy or degeneracy of the genetic code, encodes the same fusion proteins as shown in the sequence listing. This also includes synthetic, as well as chemically modified oligonucleotides, or olio nucleotides incorporating modified nucleotides such as morpholino linkages. Example of nucleic acids that can be actively transported by the methods and composition of the invention include, for example: siRNAs, ssRNA, dsRNAs, microRNAs, mRNAs, siRNAs, lncRNAs, circular RNAs, self-replicating or amplifying RNAs, chemically modified RNAs, asRNA, shRNA, and linear or circular DNA, asDNA.

The term “nucleotide sequence encoding a peptide” encompasses a nucleotide sequence which includes only coding sequences for the polypeptide, e.g., heterologous protein, as well as a polynucleotide which includes additional coding and/or non-coding sequences. Thus, for example, the polynucleotides of the present invention may encode for a peptide, e.g., a heterologous protein, or for a peptide having a prosequence or for a protein having both a prosequence and presequence.

The polynucleotides of the present invention may also have the coding sequence fused in frame to, for example, a marker sequence which allows for identification of the polypeptide of the present invention. The marker sequence may be a GFP protein, a hexa-histidine tag to provide for purification of the fusion protein is used.

The invention also relates to vectors, including but not limited to, expression vectors comprising the polynucleotides encoding the fusion proteins of the present invention. Types of vectors for expression for proteins and fusion proteins are well known in the art. In one embodiment, the vector is an expression vector for protein expression in Saccharomyces. Yeast expression vectors are commercially available from manufacturers.

The present invention also relates to methods of making and using these Saccharomyces-generated EVs. In one embodiment, the methods of making the yEVs of the present invention comprise introducing into the Saccharomyces the expression vector encoding one or more heterologous proteins related systems of actively transport therapeutic or other nucleic acid compositions into the yEV, or a fragment or variant thereof, of the present invention to generate a host Saccharomyces cell. The host cell is then cultured under conditions to permit protein production from the vector encoding the heterologous protein. In one embodiment, the host cells of the present invention Saccharomyces cerevisiae or Saccharomyces boulardii.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition). The pharmaceutical acceptable carrier may comprise any conventional pharmaceutical carrier or excipient. The choice of carrier and/or excipient will to a considerable extent depend on factors such as the particular mode of administration, the effect of the carrier or excipient on solubility and stability, and the nature of the dosage form.

Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

As used herein, a “therapeutically effective amount” includes an amount of yEVs containing a quantity of nucleic acids for treating a disease or condition for which a therapeutic nucleic acid would be beneficial. In some embodiment a therapeutically effective amount is based on the quantity of yEVs containing a quantity of nucleic acids, or the quantity of nucleic acids in a given quantity of yEVs. As also used herein, a “therapeutic nucleic acid” include nucleic acid compositions that are used to treat a disease or condition.

Culture conditions for culturing yeast host cells are well-known in the art. The continued culture of the host cell will permit production and secretion of the EVs into the cell culture environment, where they can be isolated from culture. Methods of isolating extracellular vesicles, such as exosomes, from cell culture media are well-known in the art and are reviewed in Li, P. et al., Theranostics, 7 (3): 789-804 (2017), which is incorporated by reference herein. Generally speaking, methods of isolating the EVs from culture include but are not limited to ultracentrifugation methods, size-based exclusion methods, immunoaffinity capture-based methods, precipitation methods, microfluidics-based methods or some combination thereof.

The route of administration of the yEVs includes, but is not limited to, topical, transdermal, intranasal, rectal, oral, subcutaneous, intravenous, intraarterial, intramuscular, intraosseous, intraperitoneal, epidural and intrathecal as disclosed herein. In one example yEVs may be derived or isolated from a GRAS and/or probiotic yeast cell, such as Saccharomyces cerevisiae, and preferably Saccharomyces boulardii. For example, Saccharomyces boulardii probiotics, releasing wild type exosomes, have been shown to diminish disease severity by reducing the expression of inflammatory cytokines and stimulating the expression of anti-inflammatory cytokines in multiple organs including the lungs and cardiovascular system. Saccharomyces boulardii (Sb) cells also have low immunogenicity and positively modulate host immune response in the presence of additional antigens. Cultivation of Sb is fast, low-cost, and easy to scale up using established procedures. Finally, the lipids present in EVs are natural and thus not likely to be cytotoxic when used therapeutically unlike artificial lipids frequently used to package mRNA for vaccines.

In specific embodiments, the oral administration of the yEVs includes administering engineered yeast, producing the yEVs, as a probiotic. As used herein, a probiotic is a microorganism, such as a bacteria or yeast, generally recognized as safe for human or animal consumption. The probiotics of the present invention may or may not have additional health benefits to the consumer. In specific embodiments of the present invention, the probiotics is a Saccharomyces cerevisiae or a Saccharomyces boulardii. For example, Saccharomyces boulardii probiotics, releasing wild type exosomes, have been shown to diminish disease severity by reducing the expression of inflammatory cytokines and stimulating the expression of anti-inflammatory cytokines in multiple organs including the lungs and cardiovascular system. Saccharomyces boulardii cells also have low immunogenicity and positively modulate host immune response in the presence of additional antigens. Sb is well established for genetic manipulation which allows the present inventors to engineer the Sb strain for expression and loading of specific therapeutic compositions in exosomes. Finally, Cultivation of Sb is fast, low-cost, and easy to scale up using established procedures The probiotic used in the methods of administering will be engineered to produce the SGEVs of the present invention.

The terms “comprises”, “comprising”, are intended to have the broad meaning ascribed to them in U.S. Patent Law and can mean “includes”, “including” and the like.

The invention now being generally described will be more readily understood by reference to the following examples, which are included merely for the purposes of illustration of certain embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Overview and Experimental Design

The nucleic acid specificity of the SID1 family of proteins may vary depending on experimental conditions. To assess the substrate specificity of SIDT1 and SIDT2 transporters expressed in the membrane of yEVs, Applicants tested various nucleic acids types, including ssDNA, mRNA, siRNA and dsRNA for their ability to be transported into yEVs expressing various nucleic acid transporters.

To determine the proper orientation and conformation of the SIDT proteins in yEVs membrane to load nucleic acids into the yEV lumen, various SidT gene constructs were fused with either full length Sur7 gene (encoding 4 transmembrane spans with C-terminus on the luminal side of the yEV membrane) which placed N-terminus of SIDT protein in the yEV lumen or were fused to a truncated (3 transmembrane spans with C-terminus on the outside of the yEV membrane) yeast Sur7 gene (FIG. 1). In addition, the SIDT1 gene was fused to the N-terminus of Sur7 full length gene (Sur7f) placing the N-terminus of the SIDT1 protein outside the yEVs or fused to the C-terminus of a truncated SUR7 for the same orientation of SIDT1. Fusion of SidT1 gene with N-terminus of full length Sur7 protein allowed for the unrestrained folding of the N-terminal domain of SidT1 which plays an active role in the interaction of the transporter with nucleic acids.

Conditions for function of RNA pumps are not well understood, but from their it was shown that slightly acid pH enhances the efficiency of nucleic acid transport. It is also not clear whether SidT1 and SidT2 require ATP as energy source but there is some indication that ATP may be required for nucleic acid transport. Zinc was known to be present in crystal structure of SidT homologs, thus Zn may require for proper function of RNA pumps. Thus, Applicants choose to assay RNA pump activity using a buffer containing MES pH 5.5, 150 mM NaCl, 10 mM MgCl₂, 10 μM ZnCl₂ buffer with 10 mM ATP. yEVs are stable in such buffer for several days. SidT1 and SidT2 expressing yEVs were incubated with nucleic acid substrates (0.5 μM) for 1 h. The reaction was quenched by addition of RNase A (Thermo) or DNaseI (NEB) to the reaction mix. Then, incubation was continued for another 1 h. Digested nucleic acids and protein nucleases were removed by buffer exchange using spin-columns. Thus, only nucleic acids that are protected by yEVs membranes remain in the prep. The nucleic acids-loaded yEVs were used in subsequent experiments such as modified nucleic acid fluorescence measurements or qPCR. In each experiment, wild-type yEVs were used as negative control.

To measure the concentration of fluorescently labeled nucleic acids, serial dilutions of DNA or RNA of known concentrations were used to build calibration curves. Calibration curves were run in parallel with each nucleic acid uptake experiment.

### Example 2: Demonstration of ssDNA Uptake by yEVs

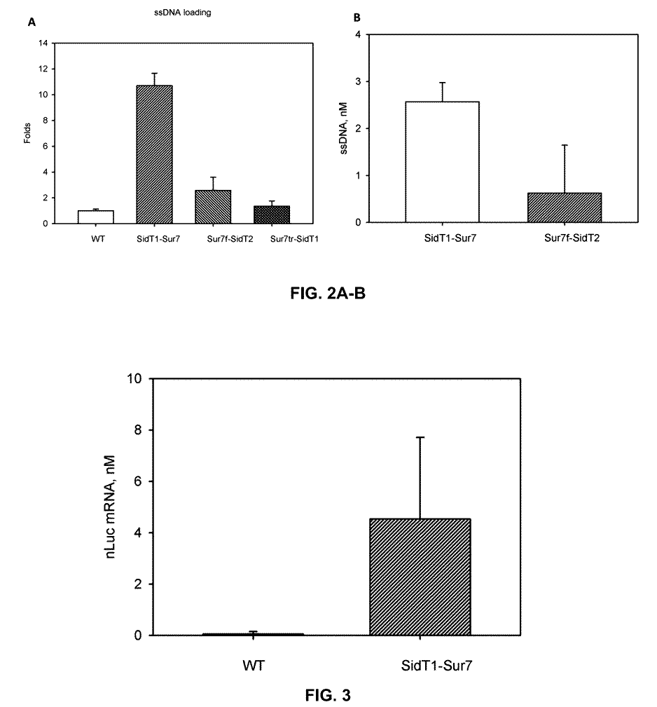

To demonstrate ssDNA uptake, Applicants used chemically synthesized DNA oligonucleotide eGFP-Alexa488 end labeled with the fluorescent dye, Alexa488 (TTGCCGGTGGTGGAGATGAA) (SEQ ID NO. 11). yEVs were incubated with eGFP-Alexa488 ssDNA, treated with DNase, and free nucleotides were removed by spin-column. Remaining fluorescence was measured in a plate reader using excitation λ 488 nm, emission λ 515 nm with cut-off filter of λ 495 nm. Results are shown on FIG. 2A. Out of all evaluated yEVs only SidT1-Sur7 yEVs demonstrated substantial uptake of ssDNA nucleotide which was 10 times background level. DNA concentration in these yEV samples was 2.6±0.7 nM (FIG. 2B), while yEVs concentration was 1.6×10¹⁰±5.2×10⁹. Thus, each yEV particle in these samples contained about 24 ssDNA molecules.

Transported nucleic acids using yEVs expressing the Sur7f-SidT2 construct, while exceeding background level, had weaker fluorescent DNA signals than yEVs expressing the SidT1-Sur7 nucleic acid transporter. The finding that only yEVs expressing nucleic acid transporters with their nucleic acid-binding domains on the outside surface of the yEV facilitated nucleic acid transport into the yEV lumen suggests that this orientation is functional and supports proper orientation of the transporters in the membrane.

### Example 3: Demonstration of mRNA Uptake by SidT1-Sur7 yEVs

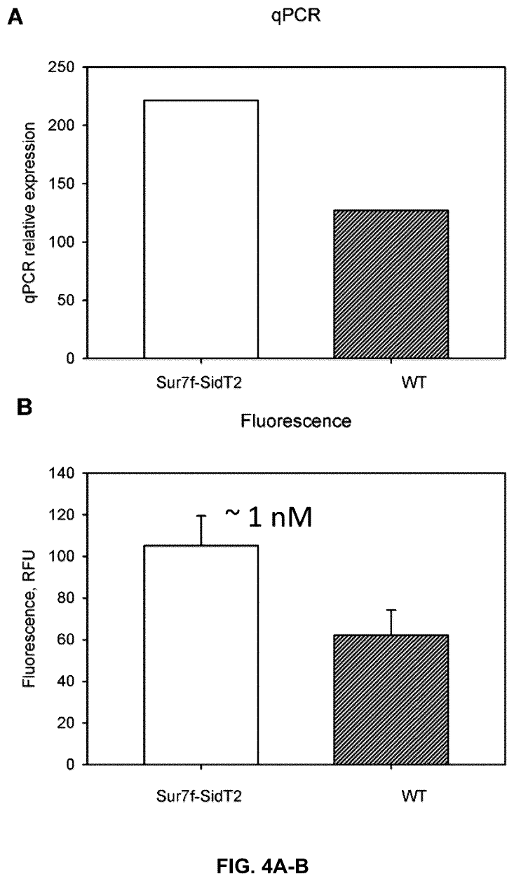

Next, Applicants evaluated the ability of SIDT1 yEVs to transport long ssRNA into the yEV lumen. Applicants observed that yEVs expressing the SIDT1-SUR7 putative nucleic acid transporter were able to transport labeled nLuc mRNA into the yEVs. As shown on FIG. 4, mRNA transported into yEVs by the SidT1-Sur7 fusion protein exceeded background levels by 8 fold (FIG. 3). As in case of ssDNA, the transport of mRNA by Sur7t-SidT1 into the yEVs did not exceed the background level further indicating that a fusion of the SidT1 gene to the N-terminus of the full length Sur7 protein resulted in the proper membrane orientation and protein conformation for the SidT1 protein to transport nucleic acids into yEVs.

### Example 4: Demonstration of mRNA Uptake by Sur7f-SidT2 EVs

The transport of ssRNA into yEVs was also evaluated for yEVs expressing the SIDT2 protein. In the mammalian cells the SidT2 protein is present in lysosomal membranes and participates in the transport of nucleic acids from the cytoplasm into the lysosomal lumen. The N-terminal part of SidT2 is localized inside lysosomes, and main cytosolic domain directly involved in the binding of nucleic acids is localized between first and second transmembrane domains of SidT2 on the outside of lysosomal membrane, unlike its location in the SidT1 protein at the very N-terminus of the protein. To reproduce the corresponding orientation of SidT2 protein in the membrane of yEVs Applicants fused SidT2 with C terminal of full-length (4 transmembrane spans with the N- and C-terminus on the luminal side of the yEV membrane) version of Sur7 anchor protein. To determine mRNA uptake by Sur7f-SidT2 construct Applicants employed two independent approaches: qPCR analysis of the amount of NLuc-mRNA loaded into yEVs and the assessment of fluorescently labeled mRNA uptake.

First, total RNA was extracted from wild-type and Sur7f-SidT2 yEVs after mRNA transport assays and used for qRT-PCR reactions. (FIG. 4A) Yeast tdh3 mRNA, which is highly enriched in yEVs, was used as reference standard for the qPCR reactions. As shown on FIG. 4A, the amount of nLuc mRNA transported into Sur7f-SidT2 yEVs was two-fold greater than that transported into wild-type yEVs. The presence of detectable amounts of mRNA in WT yEVs is most likely explained by partial protection of adhered mRNA molecules from nuclease digestion on the surface of yEVs.

Second, Applicants evaluated the transport of mRNA into yEVs by incubating wild-type or Sur7f-SidT2 expressing yEVs with nLuc-mRNA labeled with BODIPY488. As shown in FIG. 4B, mRNA loading was 2-fold greater for yEVs expressing Sur7f-SidT2 than for wild-type yEVs. Thus, we conclude that SIDT2 expressed in yEVs membranes are also capable of transporting mRNA into yEVs when the SidT2 transporter is folded into the yEV membrane in the proper orientation as described in the Sur7f-SidT2 construct.

### Example 5: Demonstration of dsRNA Uptake by yEVs Expressing Nucleic Acids Transporter Proteins

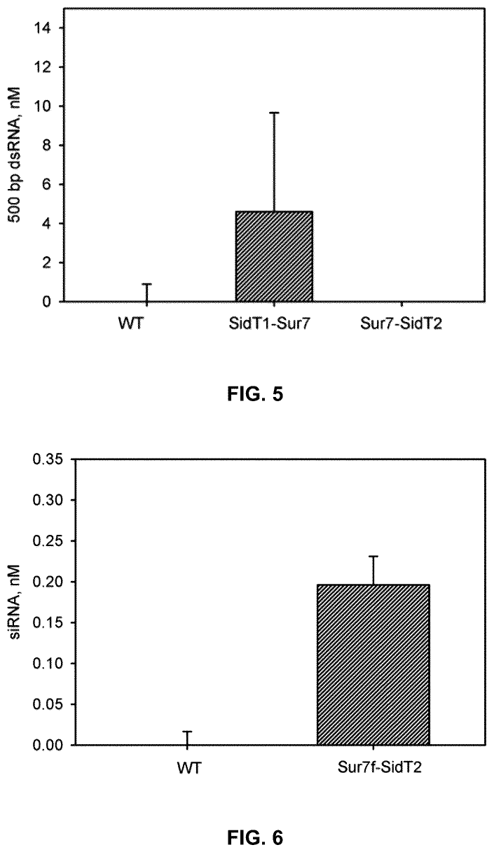

To evaluate the ability of yEVs expressing SIDT1 or SIDT2 to transport dsRNA into yEVs, Applicants used dsRNA with the length of 500 bp as the substrate. Fluorescently labeled dsRNA was obtained by in vitro transcription in presence of BODIPY488-UTP using bi-T7promoter template. Purified dsRNA was incubated with SIDT1-Sur7 and Sur7f-SIDT2 expressing yEVs. As noted in FIG. 5, only the SIDT1-SUR7 construct exhibited dsRNA transport into yEVs while the loading of dsRNA into SUR7-SIDT2-expressing yEVs did not differ from wild-type yEVs. The concentration of dsRNA contained in the SidT1-Sur7 yEVs was in the range of 2-10 nM.

### Example 5: Demonstration of siRNA Uptake by yEVS Expressing SIDT2 Proteins

Only weak interaction of SID1-family proteins with short siRNA molecules has been demonstrated only in the presence of extremely high concentrations of siRNA. Applicants sought to investigate whether siRNA could also be loaded into yEVs expressing SIDT2 transporter. Wild-type or Sur7f-SIDT2 yEVs were incubated with siRNA labeled with Cy3 fluorescent dye (Sigma SIC004). siRNA transport via Sur7f-SidT2 yEVs with a final concentration siRNA in yEVs of 0.2 nM achieved. (FIG. 6). Again, it is noted that the SUR7f-SIDT2 construct presents the proper orientation of the SIDT2 protein to facilitate nucleic acid uptake.

In conclusion, we demonstrated that variety of nucleic acids could be loaded into yEVs engineered to express the SID1-family proteins embedded in the membrane. The loading capacity of 5×10¹¹ yEVs is summarized in Table 3.

### Example 6: Materials and Methods

S. boulardii strain design and construction: To create S. boulardii strains expressing the yEV membrane localized nucleic acid pump proteins (GOI) various nucleic acid transporters constructs (SEQ ID NO's. 7-9) were fused to the C-terminus of a full or truncated version of the yEV membrane protein, SUR7 gene or to the N-terminus of Sur7 protein (SEQ ID NO. 1). Wild-type S. boulardii was transformed with linear dsDNA segments including the Sur7-GOI or the GOI-Sur7 expressing cassette consisting of a 5′ TDH3 gene promoter, genes of interest, a CYC1 3′ terminator and a geneticin-resistance gene flanked on its 5′ and 3′ ends by DNA sequences homologous to sequences from YPRCt3 locus on XVI chromosome. The dsRNA fragments were synthesized by Genscript. Yeast transformation was performed by electroporation following the protocol described by Benatuil et al (2010).

yEV isolation: Overnight cultures of S. boulardii were diluted 100 times with YPD medium. Cultures were then incubated for 24 h at 30° C. with shaking (200 rpm). For yEV isolation, cells and debris were removed by centrifugation at 3500×g for 35 min. yEVs were then concentrated from the supernatant using a tangential flow filtration device (Pall) with 300 kD membrane to a yEV concentration approximately 5×10¹¹ yEVs/mL. Isolated yEVs were aliquoted and stored in PBS buffer at −80° C.

yEV quantification: The concentration of yEVs was measured by Nanoparticle Tracking Analysis (NTA) using a Particle Metrix NTA. All samples were diluted in water to a final volume of 1.5 mL. Proper measurement concentrations were obtained by diluting samples until a concentration of 140-200 particles/frame was achieved. For each measurement, three cycles of measurements were performed by scanning 11 positions, each cycle was carried out under the following settings: Focus: autofocus; Camera sensitivity for all samples: 75; Shutter: 80; Scattering Intensity: 30; Cell temperature: 25° C. To obtain the number of yEVs per mL the video recordings were analyzed by the equipment software.

Buffer exchange of yEV preps: Both fast buffer exchange of EVs preps was performed by column gel-filtration using PCR Kleen™ Spin Columns (Bio-Rad, 7326300). 50-100 μL of yEVs suspension was applied to the center of the column prepared by manufacturer recommendations. Columns were immediately spun at 800×g for 2 min. Samples were collected at the bottom of 1.5 mL centrifuge tubes.

mRNA nLuc IVT: The nLuc construct (SEQ ID NO. 6) was used for in vitro RNA transcription of both dsRNAs and nLuc mRNA. The nLuc gene sequence was codon optimized for expression of in human cell lines. Immediately after the T7 promoter, we placed highly efficient Kozak sequence. To simplify enzyme activity assays, the nLuc coding sequence included a mammalian signal peptide that allows secretion of nLuc into culture media. To stabilize nLuc mRNA in the cells, an RNA stabilizer sequence was added at 3′ of the gene. nLuc mRNA was synthesized by in vitro transcription (IVT) using HiScribe® T7 ARCA mRNA Kit (with tailing) (NEB) using XbaI-linearized pLuc template.

Luciferase activity: To quantify transfection and translation efficiency NanoLuc luciferase was used as reporter. Nano-Glo® Luciferase Assay System (Promega) was used for detection of nLuc activity in cell supernatants.

dsRNA synthesis: dsRNAs were produced by in vitro transcription of a PCR generated DNA template containing the T7 promoter sequence added to the 5′ and 3′ end of the coding sequence to drive expression in both directions. Templates were generated by add-on PCR using an nLuc cDNA. PCR primers were designed by addition of the T7 promoter sequence (TAATACGACTCACTATAGGG) (SEQ ID NO. 12) to the 5′ end of reverse oligonucleotide while forward oligonucleotide has already contained T7 promoter sequence of nLuc construct. dsRNA was synthesized by in vitro transcription (IVT) using HiScribe® T7 High Yield RNA Synthesis Kit (NEB). Fluorescently labeled dsRNA was prepared using the NEB protocol for RNA synthesis with modified nucleotides. In these reactions UTP was partially replaced with ChromaTide™ Alexa Fluor™ 488-5-UTP (Thermo). Synthesized RNA was purified using spin column method with Monarch RNA Cleanup kit (NEB #T2040)

Human cell culture: Human lung cells (H1299 cells) (ATCC CRL-5803) were maintained in RPMI-1640 medium supplemented with 10% fetal bovine serum (FBS; Gibco, Carlsbad, CA; #26140) and 100 U/mL penicillin/streptomycin (Invitrogen, Carlsbad, CA; #15140) and grown at 37° C. and 5% CO₂.

### TABLES

TABLE 1
Sequences of the PCR oligonucleotides

| Oligo | Sequence | Purpose |
|---|---|---|
| T7-for | TACTGGCTTATCGA | Forward |
| | AATTAATACGA | oligo |
| | (SEQ ID NO. 13) | for dsRNA |
| | | production |
| T7200- | TCTAGATAATACGA | To 200 bp |
| rev | CTCACTATAGGATT | dsRNA |
| | CTGAAACAAACTGG | |
| | ACA | |
| | (SEQ ID NO. 14) | |
| T7 500- | TCTAGATAATACGA | To 500 bp |
| rev | CTCACTATAGGTTC | dsRNA |
| | CACAGGGTCCCTGT | |
| | TAC | |
| | (SEQ ID NO. 15) | |

TABLE 2
Yeast strains design and construction

| Plasmid | Construct/strain | Origin |
|---|---|---|
| pLuc | pUC54 encoding gene of | Genscript, this work |
| | nLuc luciferase | |
| Yeast strains | | |
| S. boulardii WT | | ATCC |
| SIDT1 - Sur7 | SIDT1/ GGGS3-linker/Sur7 | This work |
| Sur7f-SIDT2 | Sur7/GGGS3-linker/SIDT2 | This work |
| Sur7tr-SIDT1 | Sur7 truncated/GGGS3- | This work |
| | linker/SIDT1 | |
| Sur7tr-SIDt2 | Sur7 truncated/GGGS3- | This work |
| | linker/SIDT2 | |

TABLE 3
The amount of nucleic acids loaded by 5 × 10¹¹ yEVs expressing
nucleic transporter proteins fused with Sur7 yEVs membrane protein.

| | SidT1-Sur7 construct | Sur7f-SidT2 construct |
|---|---|---|
| | (SEQ ID NO. 7) | (SEQ ID NO 9) |
| ssDNA | 2.5 ± 1 nM | 0.5 ± 1 nM |
| ssRNA (mRNA) | 5 ± 4 nM | 1 ± 0.5 nM |
| dsRNA | 4 ± 4 nM | NT |
| siRNA | NT | 0.2 ± 0.1 nM |

### REFERENCES

1. Aizawa S, Fujiwara Y, Contu V R, Hase K, Takahashi M, Kikuchi H, et al. Lysosomal putative RNA transporter SIDT2 mediates direct uptake of RNA by lysosomes. Autophagy. 2016; 12 (3): 565-78.
2. Antoniewski C. Visitor, An Informatic Pipeline for Analysis of Viral siRNA Sequencing Datasets. Vol. 721, Methods in Molecular Biology. 2011. 123-142 p.
3. Beck A, Fecher-Trost C, Wolske K, Philipp S E, Flockerzi V, Wissenbach U. Identification of Sidt2 as a lysosomal cation-conducting protein. FEBS Lett. 2017; 591 (1): 76-87.
4. Beilharz T, Egan B, Silver P A, Hofmann K, Lithgow T. Bipartite signals mediate subcellular targeting of tail-anchored membrane proteins in Saccharomyces cerevisiae. J Biol Chem [Internet]. 2003; 278 (10): 8219-23.
5. Bleackley M R, Dawson C S, Anderson M A. Fungal Extracellular Vesicles with a Focus on Proteomic Analysis. Proteomics. 2019; 19 (8): 1-14.
6. Da Silva R P, Puccia R, Rodrigues M L, Oliveira D L, Joffe L S, César G V., et al. Extracellular vesicle-mediated export of fungal RNA. Sci Rep. 2015; 5:1-12
7. Dawson C_{S}, Garcia-Ceron D, Rajapaksha H, Faou P, Bleackley M R, Anderson M A. Protein markers for Candida albicans EVs include claudin-like Sur7 family proteins. J Extracell Vesicles [Internet]. 2020; 9 (1).
8. Egli M, Manoharan M. Re-Engineering RNA Molecules into Therapeutic Agents. Acc Chem Res. 2019; 52 (4): 1036-47.
9. Elferink H, W. H. C. Titulaer, M. G. N. Derks, G. H. Veeneman, F. P. J. T. Rutjes, T. J. Boltje, Chem. Eur. J. 2022, 28, e202103910.
10. Grossen P, Portmann M, Koller E, Duschmalé M, Minz T, Sewing S, et al. Evaluation of bovine milk extracellular vesicles for the delivery of locked nucleic acid antisense oligonucleotides. Eur J Pharm Biopharm. 2021; 158 (August 2020): 198-210.
11. Hase K, Contu V R, Kabuta C, Sakai R, Takahashi M, Kataoka N, et al. Cytosolic domain of SIDT2 carries an arginine-rich motif that binds to RNA/DNA and is important for the direct transport of nucleic acids into lysosomes. Autophagy [Internet]. 2020; 16 (11): 1974-88.
12. Haspel N, Zanuy D, Nussinov R, Teesalu T, Ruoslahti E, Aleman C. Binding of a C-end rule peptide to the neuropilin-1 receptor: a molecular modeling approach. Biochemistry. 2011 Mar. 15; 50 (10): 1755-62. doi: 10.1021/bi101662j. Epub 2011 Feb. 14. PMID: 21247217; PMCID: PMC3051018
13. Jonas S, Izaurralde E. Towards a molecular understanding of microRNA-mediated gene silencing. Nat Rev Genet [Internet]. 2015; 16 (7): 421-33.
14. Nguyen T A, Smith B R C, Elgass K D, Creed S J, Cheung S, Tate M D, et al. SIDT1 Localizes to Endolysosomes and Mediates Double-Stranded RNA Transport into the Cytoplasm. J Immunol. 2019; 202 (12): 3483-92.
15. O'Brien K, Breyne K, Ughetto S, Laurent L C, Breakefield X O. RNA delivery by extracellular vesicles in mammalian cells and its applications. Nat Rev Mol Cell Biol [Internet]. 2020; 21 (10): 585-606.
16. Oliveira D L, Nakayasu E S, Joffe L S, Guimarães A J, Sobreira T J P, Nosanchuk J D, et al. Characterization of yeast extracellular vesicles: Evidence for the participation of different pathways of cellular traffic in vesicle biogenesis. PLOS One. 2010; 5 (6): 1-13.
17. Parker S. Functional Genomics of non-coding RNA in Saccharomyces cerevisiae A thesis submitted to the University of Manchester for the Faculty of Biology, Medicine and Health. 2020
18. Pratt A J, Rambo R P, Lau P W, MacRac I J. Preparation and characterization of the extracellular domain of human Sid-1. PLOS One. 2012; 7 (4)
19. Rizzo J, Taheraly A, Janbon G. Structure, composition and biological properties of fungal extracellular vesicles. microLife. 2021; 2 (June): 1-13.
20. Schekman R. Genetic and biochemical analysis of vesicular traffic in yeast. Curr Opin Cell Biol. 1992; 4 (4): 587-92.
21 Takahashi M, Contu V R, Kabuta C, Hase K, Fujiwara Y, Wada K, et al. SIDT2 mediates gymnosis, the uptake of naked single-stranded oligonucleotides into living cells. RNA Biol 2017; 14 (11): 1534-43.
22 Zamek-Gliszczynski M J, Hoffmaster K A, Nezasa K I, Tallman M N, Brouwer Yang R, Liu P, Pan D, Zhang P, Bai Z, Xu Y, Wang L, Yan J, Yan Y, Liu X, Yang M. An Investigation on a Novel Anti-tumor Fusion Peptide of FSH33-53-IIKK. J Cancer. 2016 May 24; 7 (8): 1010-9. doi: 10.7150/jca.14425. PMID: 27313792; PMCID: PMC4910594.
23. Benatuil, L., Perez, J. M., Belk, J., and Hsieh, C. M. (2010). An improved yeast transformation method for the generation of very large human antibody libraries. Protein Eng Des Sel 23 (4), 155-159. doi: 10.1093/protein/gzq002
24. Duxbury M S, Ashley S W, Whang E E. RNA interference: a mammalian SID-1 homologue enhances siRNA uptake and gene silencing efficacy in human cells. Biochem Biophys Res Commun. 2005 Jun. 3; 331 (2): 459-63. doi: 10.1016/j.bbrc.2005.03.199. PMID: 15850781.
25. Feinberg, Evan H., and Craig P. Hunter. “Transport of dsRNA into cells by the transmembrane protein SID-1.” Science 301.5639 (2003): 1545-1547.
26. Hase K, Contu V R, Kabuta C, Sakai R, Takahashi M, Kataoka N, et al. Cytosolic domain of SIDT2 carries an arginine-rich motif that binds to RNA/DNA and is important for the direct transport of nucleic acids into lysosomes. Autophagy [Internet]. 2020; 16 (11): 1974-88.
27. Li W, Koutmou K S, Leahy D J, Li M. Systemic RNA Interference Deficiency-1 (SID-1) Extracellular Domain Selectively Binds Long Double-stranded RNA and Is Required for RNA Transport by SID-1. J Biol Chem. 2015 Jul. 31; 290 (31): 18904-13. doi: 10.1074/jbc.M115.658864. Epub 2015 Jun. 11. PMID: 26067272; PMCID: PMC4521010.
28. Qian, D., Cong, Y., Wang, R. et al. Structural insight into the human SID1 transmembrane family member 2 reveals its lipid hydrolytic activity. Nat Commun 14, 3568 (2023).
29. Shih J D, Fitzgerald M C, Sutherlin M, Hunter C P. The SID-1 double-stranded RNA transporter is not selective for dsRNA length. RNA. 2009 March; 15 (3): 384-90. doi: 10.1261/rna.1286409. Epub 2009 Jan. 20. PMID: 19155320; PMCID: PMC2657005.
30. Song, Yingying, et al. “The functions of SID1 transmembrane family, member 2 (Sidt2).” The FEBS Journal 290.19 (2023): 4626-4637
31. Vader, P., van der Aa, L. J., Engbersen, J. F. J., Storm, G., & Schiffelers, R. M. (2010). A method for quantifying cellular uptake of fluorescently labeled siRNA. Journal of Controlled Release, 148 (1), 106-109. doi: 10.1016/j.jconrel.2010
32. Zheng, Le, et al. “Cryo-EM structures of human SID-1 transmembrane family proteins and implications for their low-pH-dependent RNA transport activity.” Cell Research (2023): 1-4.

1. A Saccharomyces-generated extracellular vesicle (yEV) having a heterologous nucleic acid transport system anchored to the membrane of said yEV and adapted to actively transport one or more nucleic acid compositions into said yEV.

2. The yEV of claim 1, wherein said heterologous nucleic acid transport system comprises a fusion peptide having:
a first domain comprising an yEV membrane protein anchor; and
a second domain comprising nucleic acid transporter.

3. The method of claim 2, wherein a cytosolic nucleic acid-binding domain of the transporter is positioned on the outside surface of the yEV membrane.

4. The yEV of claim 2, wherein said fusion peptide comprises a linker peptide joining the yEV membrane protein anchor and the nucleic acid transporter domains, and wherein the linker peptide is adapted to allow proper conformational folding of the N- and C-terminal portions of the fusion peptide.

5. (canceled)

6. The yEV of claim 2, wherein said first domain is positioned at the N-terminal portion of the fusion peptide, and the second domain is positioned at the C-terminal portion of the fusion peptide.

7. The yEV of claim 2, wherein said first domain is positioned at the C-terminal portion of the fusion peptide, and the second domain is positioned at the N-terminal portion of the fusion peptide.

8. The yEV of claim 2, wherein said yEV membrane protein anchor comprises an yEV localized Saccharomyces cerevisiae SUR7 peptide, or other yEV-specific transmembrane protein, or a fragment or variant thereof.

9. The yEV of claim 2, wherein said wherein said yEV membrane protein anchor is selected from: SEQ ID NO's. 1, 4, or a fragment or variant thereof.

10. The yEV of claim 2, herein said wherein said yEV membrane protein anchor comprises a peptide having at least 80% sequence identity to SEQ ID NO. 1.

11. The yEV of claim 2, wherein said nucleic acid transporter is selected from: SIDT1, SIDT2, or a fragment or variant thereof.

12. The yEV of claim 11, wherein the signal peptide portion of SIDT1 or SIDT2 has been disrupted or removed.

13. The yEV of claim 2, wherein said nucleic acid transporter is selected from: SEQ ID NO's. 2, 5, or a fragment or variant thereof.

14. The yEV of claim 2, wherein said nucleic acid transporter comprises a peptide having at least 80% sequence identity to SEQ ID NO. 2, or a peptide having at least 80% sequence identity to SEQ ID NO. 5.

15. The yEV of claim 4, wherein said peptide linker comprises a peptide linker according to SEQ ID NO. 3.

16. The yEV of claim 1, wherein said heterologous nucleic acid transport system comprises a fusion peptide having:
a first domain at the N-terminal position comprising an yEV membrane protein anchor according to SEQ ID NO. 1, or a fragment or variant thereof;
a second domain at the C-terminal position comprising a nucleic acid transporter according to SEQ ID NO. 5, or a fragment or variant thereof; and
wherein said first and second domain are joined by a peptide linker.

17. The yEV of claim 1, wherein said heterologous nucleic acid transport system comprises a fusion peptide having:
a first domain at the C-terminal position comprising an yEV membrane protein anchor according to SEQ ID NO. 1, or a fragment or variant thereof;
a second domain at the N-terminal position comprising a nucleic acid transporter according to SEQ ID NO. 2; and
wherein said first and second domain are joined by a peptide linker.

18. The yEV of claim 1, wherein said heterologous nucleic acid transport system comprises a nucleic acid transport system according to SEQ ID NO. 7, 9, or a fragment or variant thereof.

19. (canceled)

20. The yEV of claim 1, wherein the nucleic acid to be transported into the yEV is selected from: DNA, RNA, a chemically modified oligonucleotide, ssDNA, ssRNA, dsRNA, siRNA, microRNAs, mRNAs, siRNAs, lncRNAs, circular RNAs, self-replicating or amplifying RNAs, chemically modified RNAs, shRNA, and linear or circular DNA, and antisense oligonucleotides, or a combination of the same.

21. (canceled)

22. The yEV of claim 1, wherein the yEV is generated from Saccharomyces cerevisiae, or Saccharomyces boulardii.

23. (canceled)

24. A pharmaceutical composition comprising a Saccharomyces-generated extracellular vesicle (yEV) of claim 1, and a pharmaceutically acceptable carrier.

25-63. (canceled)
